(19) United States
(12) Patent Application Publication
(10) Pub. No.: US 2025/0276950 A1
(43) Pub. Date: Sep. 4, 2025

(54) BIOMONOMERS AND THEIR RESPECTIVE BIOPOLYMERS FOR PAINTS AND VARNISHES

(71) Applicant: NUOL Green Chemistry LLC, Little Falls, MN (US)

(72) Inventor: Setsuo Sato, Piracicaba (BR)

(21) Appl. No.: 18/596,175

(22) Filed: Mar. 5, 2024

(30) Foreign Application Priority Data
Mar. 1, 2024 (BR) 10 2024 004247 6

(51) Int. Cl. C07C 67/02; C09D 133/06

(52) U.S. Cl. CPC C07C 67/02; C09D 133/066

(57) ABSTRACT

The development of acrylated biomonomers produced from vegetable oil derivatives with a high degree of unsaturation allows the production of esters with a high content of epoxy precursor for the production of acrylated monomers with a high content of unsaturations capable of producing excellent varnishes. Starting with common and widely available oils such as soybean and similar oils, removing the saturated part, thus generating materials with a high degree of unsaturation similar to fatty materials derived from linseed oil. These biomonomers and their respective biopolymers in applications have demonstrated excellent performance, durability, are odorless, cure quickly, are biodegradable and economically more attractive.

The use of products of natural origin has been greatly encouraged due to their environmental benefits, although the performances are not the same when compared to traditional products that have been in use for a long time. Truly innovative solutions are a challenge for the industry, requiring investment, time, resilience and especially an educational system that creates an appropriate environment for innovation.

Materials that are produced from wood used externally or internally require protection to guarantee their preservation and aesthetics. For this purpose, varnishes produced from acrylic monomers of petrochemical origin are used, the processes of which consume a lot of energy, generate emissions and are not biodegradable. The need for more environmentally friendly materials is an urgent need in this market segment.

The invention is based on the development of acrylated monomers produced from derivatives of vegetable oils with a high degree of unsaturation, which allows the production of esters with a high content of epoxy precursor for the production of acrylated monomers with a high content of unsaturations capable of producing excellent varnishes.

Fatty materials produced from linseed or tung oil have a high degree of unsaturation compared to vegetable oils such as soybean, corn, peanut, canola, sunflower; however, they are scarce and expensive when compared to them. In this invention we start from common and widely available oils such as soybean and similar oils, removing the saturated part, thus generating materials with a high degree of unsaturation similar to fatty materials derived from linseed oil.

These biomonomers in applications have demonstrated excellent performance, durability, are odorless, cure quickly, are biodegradable and economically more attractive.

These types of materials are usually produced using acrylic monomers via polymerization in petrochemical solvents. As a result, we have a process with low energy consumption, zero emissions and polymeric varnishes are biodegradable.

### STATUS QUO

Journal Polym Environ (2012) 20:1063-1074 mentions the use of epoxidized and acrylated linseed oil as an ultraviolet curing varnish for application to wood, demonstrating its excellent performance in terms of adhesion, mechanical resistance, solvent resistance and coating capacity.

One of the products on the market is acrylated epoxidized methyl ricinoleate produced from castor oil, as shown in FIG. 1.

The main negative point of materials of plant origin is that they are not “pure”, that is, they have other components that are inert and do not form the desired components and in applications they end up as incompatible materials, migrating and causing problems.

### DESCRIPTION OF THE INVENTION

In this development, the first step was to obtain fatty material containing a maximum of 5% unsaturated material, mainly the palmitic and stearic fatty chains.

From this material we built the desired molecules, mainly aiming for the best chemical structure, molecular weight, number of unsaturations according to the target applications, as shown in FIGS. 2 to 4.

In this way, the following are described:

1. Production and use of the chemical structures as shown in FIGS. 2, 3 and 4 and similar ones that are produced via esterification and/or transesterification using the alcohols methanol, ethanol, isopropanol, butanol, isobutanol, cyclohexanol, ethylhexanol, neopentylglycol, trimethylolpropane, pentaerythritol, propylene glycol, dipropylene glycol, ethylene glycol, diethylene glycol, triethylene glycol, glycerin, polyglycerin-3, polyglycerin-6.

Production and use, according to item 1 in which the fatty acids are those derived from soybean, canola, corn, rapeseed, peanut, palm oils which are enzymatically hydrolyzed, and the saturated fatty acids are separated via crystallization with or without solvent so that at the end the unsaturated part has a maximum of 6% saturated fatty acids.

3. Products produced according to steps 1, 2, 3, 4, 5 and 6 in which the products are biomonomers for the production of acrylic biopolymers whose technical performance is equivalent to petrochemical acrylic polymers with the advantages of being biodegradable, non-toxic and can be handled without risk to the worker, and are economically more attractive.

4. Products, according to item 3, biomonomers will be used as biopolymers such as varnishes for wood in indoor and outdoor applications, in the production of domestic paints, construction paints, and automotive applications.

5. Products, in accordance with item 4, which will also be used in applications for the production of food packaging and all applications where acrylic polymers of petrochemical origin are used.

### BRIEF DESCRIPTION OF FIGURES

FIG. 1 represents the structure of acrylated epoxidized methyl ricinoleate.

FIG. 2 represents one of the biomonomers according to the invention, namely epoxidized acrylate trimethylpropane trioleate/linoleate.

FIG. 3 represents one of the biomonomers according to the invention, namely acrylated epoxidized fatty ester.

FIG. 4 represents one of the biomonomers according to the invention, namely acrylated epoxidized fatty acid diester.

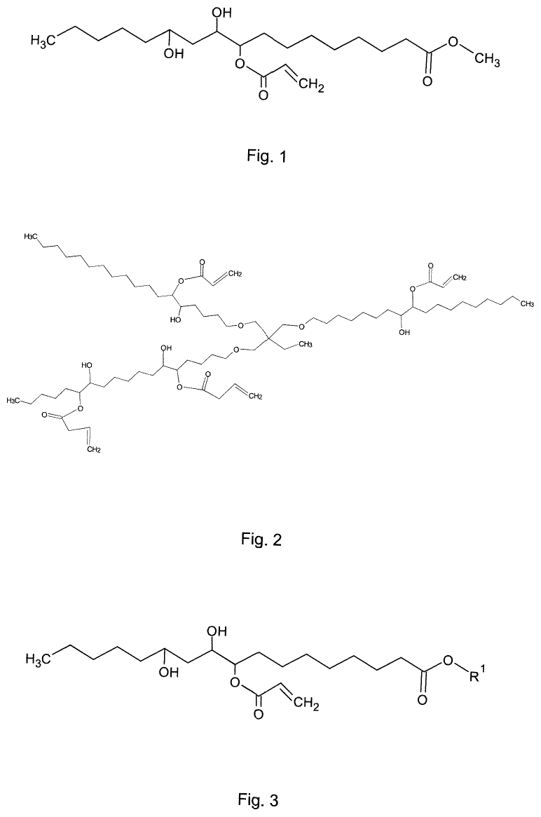

### STEPS USED IN PRODUCT PREPARATION

Selection of vegetable oils with an iodine index above 100 cg I2/100 grams of product (soy, corn, peanut, sunflower, rapeseed, canola, palm).

Enzymatic hydrolysis using lipases at a temperature of 40° C. with a minimum conversion of 97%.

Selective crystallization with separation of saturated chains, with the unsaturated phase containing a maximum of 6% saturated material (stearic and palmitic).

Esterification of fatty acids with alcohols and polyols at temperatures from 200° C. to 240° C. with a minimum 99% conversion. Methanol, ethanol, isopropanol, butanol, isobutanol, cyclohexanol, ethylhexanol, neopentyl glycol, trimethylolpropane, pentaerythritol, propylene glycol, dipropylene glycol, ethylene glycol, diethylene glycol, triethylene glycol, glycerin, polyglycerin-3, polyglycerin-6 as represented in chemical structures as R1.

Epoxidation of components via hydrogen peroxide catalyzed by performic acid with conversion of unsaturated chains at least 80%.

Opening the epoxy rings with acrylic acid or methacrylic acid.

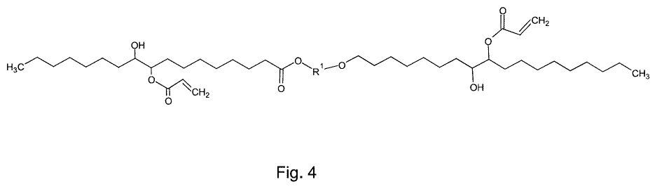

1. Production and use of chemical structures as shown in FIGS. 2, 3 and 4 and their similar characteristics characterized in that they are produced via esterification and/or transesterification using the alcohols methanol, ethanol, isopropanol, butanol, isobutanol, cyclohexanol, ethylhexanol, neopentyl glycol, trimethylolpropane, pentaerythritol, propylene glycol, dipropylene glycol, ethylene glycol, diethylene glycol, triethylene glycol, glycerin, polyglycerin-3, polyglycerin-6.

2. Production and use, according to claim 1, characterized in that the fatty acids are those derived from soybean, canola, corn, rapeseed, peanut, palm oils which are enzymatically hydrolyzed and the saturated fatty acids are separated via crystallization with or without solvent so that in the end the unsaturated part has a maximum of 6% saturated fatty acids.

3. Products produced according to steps 1, 2, 3, 4, 5 and 6 the process of claim 1 characterized in that they are biomonomers for the production of acrylic biopolymers whose technical performance is equivalent to petrochemical acrylic polymers with the advantages of being biodegradable, are non-toxic and can be handled without risk to the worker and are economically more attractive.

4. Products, according to claim 3, characterized in that the biomonomers will be used as biopolymers such as varnishes for wood in indoor and outdoor applications, in the production of domestic paints, civil construction, and automotive applications.

5. Products, according to claim 4, characterized in that they will also be used in applications for the production of food packaging and all applications where acrylic polymers of petrochemical origin are used.

6. Products produced according to steps 1, 2, 3, 4, 5 and 6 the process of claim 2 characterized in that they are biomonomers for the production of acrylic biopolymers whose technical performance is equivalent to petrochemical acrylic polymers with the advantages of being biodegradable, are non-toxic and can be handled without risk to the worker and are economically more attractive.

7. Products, according to claim 6, characterized in that the biomonomers will be used as biopolymers such as varnishes for wood in indoor and outdoor applications, in the production of domestic paints, civil construction, and automotive applications.

8. Products, according to claim 7, characterized in that they will also be used in applications for the production of food packaging and all applications where acrylic polymers of petrochemical origin are used.
